(19) United States
(12) Patent Application Publication
(10) Pub. No.: US 2025/0152785 A1
(43) Pub. Date: May 15, 2025

(54) OSTEOSYNTHESIS SYSTEM WITH BONE PLATE AND BONE ANCHOR MADE OF MAGNESIUM ALLOYS

(71) Applicant: Medical Magnesium GMBH, Aachen (DE)

(72) Inventors: Alexander Kopp, Aachen (DE); Kilian Reuss, Aachen (DE); Lennart Kölbel, Aachen (DE)

(21) Appl. No.: 18/839,509

(22) PCT Filed: Mar. 9, 2023
(86) PCT No.: PCT/EP2023/055972

§ 371 Date: Aug. 19, 2024

(30) Foreign Application Priority Data
Mar. 21, 2022 (DE) 10 2022 106 581.2

(51) Int. Cl. A61L 31/02; A61B 17/00; A61B 17/80; A61L 31/08; A61L 31/14

(52) U.S. Cl. CPC A61L 31/022; A61B 17/8057; A61L 31/086; A61L 31/088; A61L 31/148; A61B 2017/00004; A61L 2430/02

(57) ABSTRACT

An osteosynthesis system includes at least one bone plate (2), which has at least one receiving opening (8), and a bone anchor (3), which has an anchor head, the anchor head being designed to be fixed in the receiving opening by forming a form-fitting and/or frictional connection with the bone plate (2). The bone plate (2), at least in a portion including the receiving opening, and the bone anchor (3), at least in a portion including the anchor head, are each made of a magnesium alloy, the magnesium alloy of the bone anchor (3) having a different hardness to the magnesium alloy of the bone plate (2).

The invention relates to an osteosynthesis system with a bone plate, which has at least one receiving opening, and with a bone anchor, which comprises an anchor head with a connecting structure, the anchor head being designed to be fixed in the receiving opening by forming a form-fitting and/or frictional connection with the bone plate by means of the connecting structure of the anchor head.

Such a system is known from WO 00/66012 A1. The fixation can be achieved by an interaction of threads of the anchor head and of the bone plate (as internal thread of the receiving opening). These threads are not complementary, so when the threaded connection is formed, the material of one of the connection partners is deformed. This results in a high loosening torque for the threaded connection, as a result of which unintentional loosening can be avoided.

In order to achieve a deliberate deformation of the material of a connection partner in such an osteosynthesis system, the bone plate on the one hand and the bone anchor on the other hand are usually made of different materials that differ in particular in terms of hardness. A thread made of a harder material can therefore cut into the softer material of the other connection partner in a defined manner. In this regard, it is known to use different titanium alloys, for example Grade 4 on the one hand and Grade 5 on the other, for the connection partners.

The invention is based on the object of improving such an osteosynthesis system.

This object is achieved in a system according to patent claim 1. Advantageous embodiments thereof form the subject matter of the further claims and/or can be found in the following description of the invention.

According to the invention, in an osteosynthesis system comprising at least one bone plate, which has at least one receiving opening, and at least one bone anchor, which has an anchor head, the anchor head being designed to be fixed in the receiving opening by forming a form-fitting and/or frictional connection with the bone plate, it is provided for the bone plate, at least in a portion comprising the receiving opening, and the bone anchor, at least in a portion comprising the anchor head, to be each made of a magnesium alloy, the magnesium alloy of the bone anchor having a different hardness to the magnesium alloy of the bone plate. It is preferably provided for the magnesium alloy of the bone anchor to have a higher hardness than the magnesium alloy of the bone plate.

A “bone plate” is understood to be a component that is provided and designed to bear against a bone. The component can in particular be designed to be planar (flat or curved), so that a length and a width of the component are each larger, in particular at least two or five or ten times larger, than a height of the component.

A “bone anchor” is understood to be a component that is provided and designed to be fixed within a bone.

The hardness of the magnesium materials can be determined in particular as Vickers hardness (HV) according to DIN EN ISO 6507-1:2018 to -4:2018.

Magnesium or magnesium alloys, in particular magnesium-rare earth alloys, magnesium-calcium-zinc alloys and magnesium-aluminum alloys with or without the addition of yttrium, have an advantageous biocompatibility, so they can be advantageously used for an osteosynthesis system. At the same time, magnesium alloys have sufficient mechanical properties (in particular in terms of strength) to adequately support the forces typically exerted on the components of such a system during use. Another particularly relevant advantage of magnesium alloys is that they are resorbable, so that at least the magnesium component dissolves over a relatively long period of time in a defined manner through interaction with the body's own substances. This means that an implanted osteosynthesis system does not have to be removed again after the bone fracture has healed. Accordingly, it can preferably be provided for the magnesium alloy of the bone plate and/or the magnesium alloy of the bone anchor to be resorbable.

If a system according to the invention comprises multiple bone plates and/or bone anchors, it can preferably be provided for all of these components to be partially or completely made of magnesium alloys.

According to a preferred embodiment of a system according to the invention, it can be provided for the hardness of the magnesium alloy with the lower hardness to be between 40 and 80 HV or between 30 and 50 HV or between 35 and 45 HV, and/or for the hardness of the magnesium alloy with the higher hardness to be at least 85 HV.

According to a preferred embodiment of a system according to the invention, it can be provided for the magnesium alloy with the higher hardness to have a material structure achieved by cold forming. Cold forming is an advantageous way to increase the hardness of a magnesium alloy. In order to achieve the difference in hardness of the magnesium alloys according to the invention, it can then preferably be provided for the magnesium alloy with the lower hardness not to have a material structure achieved by cold forming. In principle, however, different hardnesses can be achieved in magnesium alloys by different cold forming processes, and therefore the magnesium alloy with the lower hardness can likewise have a material structure achieved by cold forming.

A particularly pronounced increase in the hardness of a magnesium alloy can be achieved by hammering, so it can preferably be provided for the magnesium alloy with the higher hardness to have a material structure achieved by hammering. The magnesium alloy with the lower hardness, on the other hand, preferably can have a material structure not achieved by hammering.

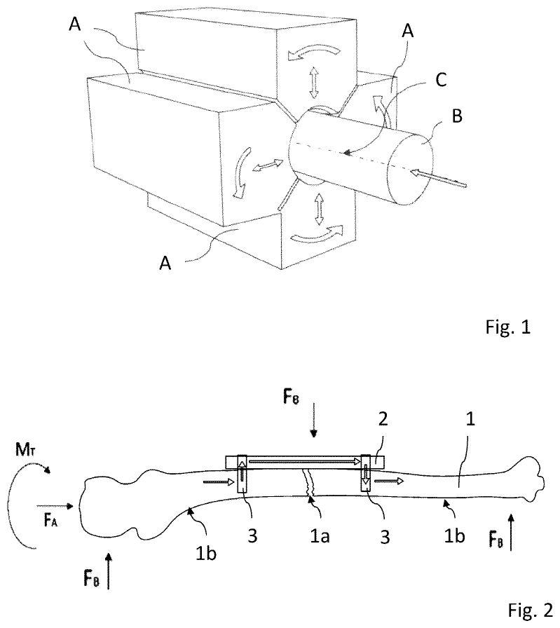

Hammering, also known as rotary die forging or rotary swaging, is characterized in that two or more tools (A), which are arranged on the circumference of a workpiece (B), exert forming strokes directed radially toward a workpiece center, while the workpiece (B) rotates about the workpiece center relative to the tools (A), as shown schematically in FIG. 1. A relative movement between the workpiece (B) and the tools (A) along a longitudinal axis (C) of the workpiece (B) allows continuous or discontinuous forming even of workpieces (B) with dimensions along the longitudinal axis (C) that are larger than the corresponding dimensions of the tools (A). The relative movement between the workpiece (B) and the tools (A) along the longitudinal axis (C) of the workpiece (B) can preferably be realized by moving (only) the workpiece (B) longitudinally axially. Alternatively or additionally, a corresponding movement of the tools (A) can also be provided, however. The strokes performed by the tools (A) can in particular be relatively short (e.g. between 0.25 mm and 3 mm, in particular between 0.3 and 1.5 mm) and performed at a relatively high frequency (e.g. 1000 per minute and more). Furthermore, it can preferably be provided for the tools (A) to surround the workpiece (B) almost completely, i.e., with only minimal distances between the tools (A), on the circumference.

According to a preferred embodiment of a system according to the invention, it can be provided for the magnesium alloy of the bone plate on the one hand and the magnesium alloy of the bone anchor on the other hand to have the same chemical composition, i.e., the chemical elements of which the magnesium alloys are mainly composed (i.e., each with a proportion of at least 0.1 wt. %; since elements with a proportion less than 0.1 wt. % are considered to be impurities according to the invention) are on the one hand the same and are also present in equal proportions (“equal” according to the invention means, with regard to the alloy proportions, with a maximum relative deviation of 10% based on the larger of the compared proportions in absolute terms. Different hardnesses of the magnesium alloys for the bone plate on the one hand and the bone anchor on the other hand can be achieved by different processing operations and/or treatments of the magnesium alloys.

The use of magnesium alloys of the same chemical composition for the bone plate on the one hand and the bone anchor on the other hand, preferably for the entire system, allows simple and cost-effective production. It can be particularly advantageous that, if necessary, proof of chemical, toxicological and biological safety or the required biocompatibility according to DIN EN ISO 10993:2016 needs to be provided for only one magnesium alloy (defined by the chemical composition within certain ranges), and the interaction of different materials does not have to be addressed with regard to certification as a medical device.

According to a preferred embodiment, the magnesium alloy of the bone plate and/or the magnesium alloy of the bone anchor can be an Mg—Y-RE-Zr alloy and particularly preferably an Mg—Y—Nd—Zr alloy, which is also known as a WE43 alloy. In particular the rare earth (RE) elements Dy, Y, Nd and Gd have low toxicity when used as alloying elements in magnesium alloys and exhibit advantageous mechanical and corrosion properties when used clinically in corresponding alloys as implant material.

The magnesium alloy of the bone plate and/or the magnesium alloy of the bone anchor can advantageously also be an Mg—Y—Nd alloy with or without the addition of Zr. In a preferred embodiment, such a magnesium alloy has an yttrium content between 3 wt. % and 5 wt. % and an Nd content between 2 wt. % and 4 wt. %.

The magnesium alloy of the bone plate and/or the magnesium alloy of the bone anchor can advantageously also comprise calcium and zinc, preferably as an Mg—Ca—Zn alloy or as an Mg—Zn—Ca alloy, each with or without the addition of Zr. The Ca and Zn contents can preferably each be less than 1 wt. % or less than 2 wt. % or less than 5 wt. %.

According to a preferred embodiment of a system according to the invention, it can be provided for the receiving opening of the bone plate in an initial state (i.e., before connection to the bone anchor for this first time) not to have a connecting structure complementary to a connecting structure of the anchor head. Alternatively, it can be provided for the anchor head in an initial state not to have a connecting structure complementary to a connecting structure of the receiving opening. Connecting the bone plate and the bone anchor therefore results in plastic deformation.

Preferably, it can be provided for the connecting structure of the anchor head, if provided, to comprise or be a thread, in particular an external thread. If the receiving opening of the bone plate in such a system according to the invention does not have a connecting structure complementary to the connecting structure or the thread of the anchor head, it can preferably be provided for it to be, in the initial state, thread-free or provided with a thread, in particular an internal thread, that is not complementary to the thread of the anchor head due to different thread parameters.

It can also be provided for the connecting structure of the receiving opening, if provided, to comprise or be a thread, in particular an internal thread. If the anchor head in such a system according to the invention does not have a connecting structure complementary to the connecting structure or the thread of the receiving opening, it can preferably be provided for it to be, in the initial state, thread-free or provided with a thread, in particular an external thread, that is not complementary to the thread of the receiving opening due to different thread parameters.

The bone anchor of a system according to the invention can preferably be designed as a bone screw and for this purpose have an anchor shaft with a shaft thread which is intended to engage or cut into bone material when the anchor shaft is (possibly only partially) positioned within an opening in a bone. This allows a connection between the bone anchor or bone screw and the bone to be achieved that is easy to achieve and at the same time securely durable. Furthermore, it can then preferably be provided for the thread of the anchor head and the shaft thread to have different thread parameters (in particular different thread leads and/or thread pitches and/or flank angles and/or core or outer diameters) at least in portions, whereby the stability of the fixation in the bone material or the necessary torque when screwing into the bone material can be reduced or controlled.

According to a preferred embodiment of a system according to the invention, it can be provided for there to be a coating on a main body made of the (respective) magnesium alloy on the bone plate, at least in a portion comprising the receiving opening, and/or on the bone anchor, at least in a portion comprising the anchor head. Such a coating can be used in particular to influence and in particular to slow down the corrosion behavior and thus possibly also the resorption behavior of the magnesium alloy of the associated main body. By coating the respective main body at least in a portion of the bone plate comprising the receiving opening and/or the portion of the bone anchor comprising the anchor head, in particular the regions of the connection between the bone plate and the anchor head, via which relatively high forces and moments are usually transmitted, can be kept stable for as long as possible.

Preferably, it can be provided for the coating to comprise or be an oxide layer, in particular a magnesium oxide layer. Such an oxide layer can particularly preferably be produced or have been produced by means of plasma electrolytic oxidation (PEO). This is a combined method from the fields of plasma technology and electrochemistry, by means of which surfaces of components made of so-called valve metals can be provided with a surface layer of an oxide ceramic (cf. WO 2015/090267 A1). Native barrier layer formers, such as magnesium, are particularly suitable as valve metals. The surface layer can be generated in particular in aqueous electrolytes. The component(s) to be oxidized can be immersed in the electrolyte as an electrode together with one or more other components that act as a counter electrode. When direct current is used, the component(s) to be coated are anodically polarized. With alternating current or bipolar pulsed currents, the component(s) to be coated are only “electrodes”, wherein the surface layer only ever forms in the current segments in which the component(s) are anodes. In the case of symmetrical pulsed or alternating currents (i.e., the same proportion of current in both directions), the components used as “electrode” as well as those used as “counter electrode” can therefore be alternately connected anodically and thus provided with a surface layer. During PEO, the component(s) to be coated initially form a purely chemically induced passive layer. The growth of this passive layer can be achieved by applying a potential between the anodically polarized component and the cathode. The passive layer of the component to be coated is locally broken down, triggering plasma-chemical solid-state reactions, the spark discharges. PEO is therefore also called “anodic oxidation by spark discharge” (ANOF). The spark discharges are not generated over a large area but only locally at the points at which the thickness of the oxide layer and thus the local electrical resistance is lowest. Since the plasma reactions thus always take place at the points of the passive layer that locally have the smallest layer thickness and cause the layer thickness to grow there, the surface is covered with a very uniform surface layer. In order to continuously break through the increasing dielectric property of the growing oxide layer with a breakdown voltage, the applied electrical potential is increased until the desired layer thickness of the surface layer is reached.

According to a preferred embodiment of a system according to the invention, it can be provided for the receiving opening and/or the anchor head to have a conical or frustoconical basic shape, whereby advantageous transmissions of forces and moments between the bone plate and the bone anchor can be realized; this applies in particular also to a non-parallel and in particular also non-coaxial orientation of the bone anchor within the receiving opening of the bone plate, i.e., when a central longitudinal axis of the bone anchor in the region of the anchor head is non-parallel or non-coaxial and thus oriented at an angle with respect to a central longitudinal axis of the receiving opening, as can regularly be the case when a system according to the invention is used.

If both the receiving opening and the anchor head have conical or frustoconical basic shapes, it can be particularly preferably provided for these basic shapes to have different conicities, i.e., the changes in diameter for the different conical basic shapes are different.

The invention also relates to a method for osteosynthesis using a system according to the invention, in which the bone plate is applied to a bone in the region of a bone fracture, and at least one, preferably both of the two bone portions separated by the bone fracture are connected to the bone plate by means of the or in each case one bone anchor, and the bone fracture is thereby supported via the bone plate. The connection of a bone portion to a bone anchor is carried out by inserting and fixing the bone anchor in the bone portion and, preferably at least partially simultaneously, by forming the form-fitting and/or frictional connection between the anchor head of the bone anchor and the bone plate.

The invention is explained in more detail below with reference to exemplary embodiments shown in the drawings. The drawings show the following, in some cases in simplified form:

FIG. 1: processing of a workpiece by hammering;

FIG. 2: an osteosynthesis system according to the invention in use;

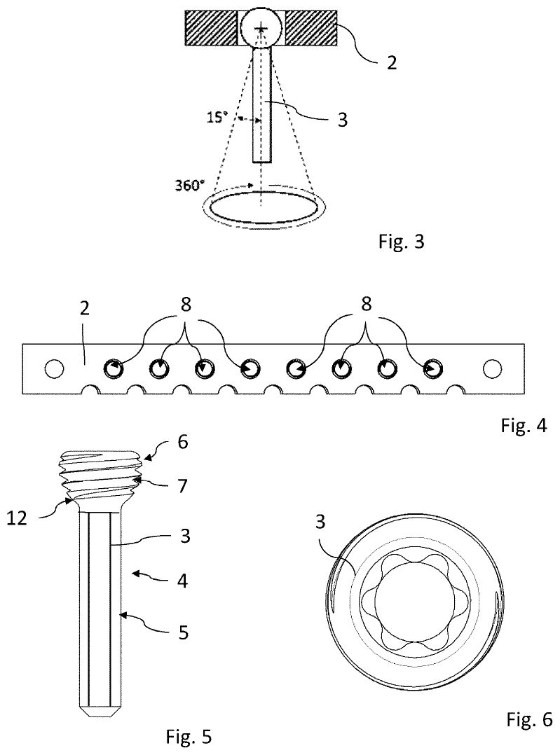

FIG. 3: an allowable angular range with regard to the orientation of a bone plate and a bone anchor in the system;

FIG. 4: the bone plate of the system in a top view;

FIG. 5: the bone anchor of the system in a side view;

FIG. 6: the bone anchor of the system in a top view;

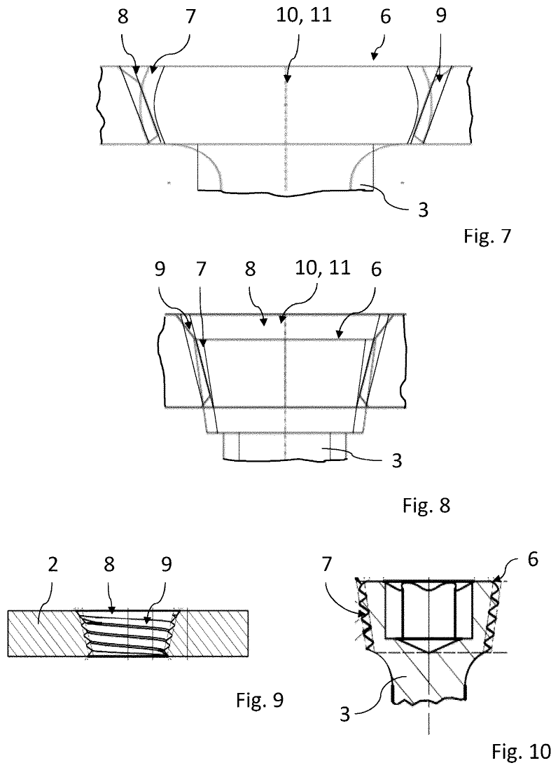

FIG. 7: the thread shapes of an internal thread of a receiving opening of the bone plate and an external thread of the bone anchor of the system;

FIG. 8: alternative thread shapes of an internal thread of a receiving opening of a bone plate and an external thread of a bone anchor of an osteosynthesis system according to the invention;

FIG. 9 the internal thread of the bone plate according to FIG. 8 in detail; and

FIG. 10: the external thread of the bone anchor according to FIG. 8 in detail.

FIG. 2 shows a system according to the invention for osteosynthesis in use, i.e., for the stabilization of two bone portions 1b of a bone 1 separated by a bone fracture 1a until the bone fracture 1a heals. The system is intended to support forces F_{A} in the longitudinal direction of the bone 1, as well as forces F_{B} in the transverse direction of the bone 1, which cause bending, as well as torsional moments M_{T} about the longitudinal direction of the bone 1. For this purpose, a bone plate 2 of the system bears against the two bone portions 1b and spans the bone fracture 1a. The bone plate 2 is firmly connected to each of the two bone portions 1b via at least one bone anchor 3. The bone anchors 3 are firmly anchored at one end in the corresponding bone portion 1b, which can preferably be achieved by screwing a shaft thread 5, which is formed in the region of an anchor shaft 4 of each bone anchor 3 (cf. FIG. 5), into a previously made bore in the bone portion 1b in question, wherein the shaft thread 5 cuts into the bone material of the respective bone portion 1b.

The bone anchors 3 are also each firmly connected to the bone plate 2 via a form-fitting and/or frictional connection. This connection is brought about by an interaction of an external thread 7, which acts as a connecting structure of each bone anchor 3 and is formed in the region of an anchor head 6 of each bone anchor 3, with an internal thread 9, which acts as a connecting structure of the bone plate 2 and is formed in the region of each of a plurality of receiving openings 8 of the bone plate 2. However, it is provided for the external threads 7 of the bone anchors 3 not to be complementary to the internal threads 9 of the bone plate 2 in the respective initial state due to different thread parameters, so that screwing the external thread 7 of a bone anchor 3 into an internal thread 9 of the bone plate 2 causes a defined plastic deformation (substantially exclusively) of the material of the bone plate 2. As a result, a particularly secure connection between the bone anchors 3 and the bone plate 2 can be realized, wherein in particular the loosening torque required for unscrewing the external thread 7 of the anchor head 6 of a bone anchor 3 from the associated internal thread 9 of the bone plate 2 is comparatively high, so that unintentional loosening is effectively prevented.

Both the bone plate 2 and all the bone anchors 3 consist entirely of resorbable magnesium alloys of the same chemical composition (e.g. magnesium WE43). In order to ensure that substantially only the magnesium alloy of the bone plate 2 is deformed deliberately when the external threads 7 of the anchor heads 6 of the bone anchors 3 are screwed into the associated internal threads 9 of the bone plate 2, it is provided for the magnesium alloy of the bone anchors 3 to have a higher hardness (e.g. approximately 20% higher) than the magnesium alloy of the bone plate 2. This is achieved despite the use of the same magnesium alloy for the bone plate 2 on the one hand and the bone anchors 3 on the other hand, in that blanks of the magnesium alloy used for the production of the bone anchors 3 were processed by hammering, whereby a corresponding increase in hardness can be realized, while the blank of the magnesium alloy from which the bone plate 2 was made was not processed by hammering.

When a system according to the invention is implemented, it cannot usually be guaranteed that the bone anchors 3 are screwed in with an exactly coaxial orientation of their central longitudinal axis 10 with respect to the central longitudinal axis 11 of the associated receiving openings 8 of the bone plate 2. In order to still ensure sufficient load-bearing capacity of the threaded connections formed between the bone anchors 3 and the bone plate 2, the receiving openings (including the internal threads 9) of the bone plate 2 and the anchor heads 6 (including the external threads 7) of the bone anchors 3 have specific, mutually adapted basic shapes which ensure sufficient load-bearing capacity at least up to a maximum deviation of 15° from the coaxial orientation (cf. FIG. 3). According to FIG. 7, a partially spherical basic shape of the anchor heads of the bone anchors can be combined with a conical basic shape of the receiving openings of the bone plate for this purpose. In the alternative embodiment according to FIG. 8, however, both the basic shape of the anchor heads of the bone anchors on the one hand and the basic shape of the receiving openings of the bone plate on the other hand are conical, but these are designed with different conicities.

In order to influence in particular the degradation behavior of the components of a system according to the invention in a targeted manner, these components can be provided at least in portions with a coating, for example an oxide layer produced by means of PEO (cf. FIG. 5).

### LIST OF REFERENCE SYMBOLS

1 Bone
1a Bone fracture
1b Bone portion
2 Bone plate
3 Bone anchors
4 Anchor shaft of bone anchor
5 Shaft thread
6 Anchor head of bone anchor
7 External thread of anchor head
8 Receiving opening of bone plate
9 Internal thread of receiving opening
10 Central longitudinal axis of bone anchor
11 Central longitudinal axis of receiving opening
12 Coating
A Tool
B Workpiece
C Workpiece longitudinal axis

1. An osteosynthesis system with a bone plate (2), which has at least one receiving opening (8), and with a bone anchor (3), which comprises an anchor head (6), the anchor head (6) being designed to be fixed in the receiving opening (8) by forming a form-fitting and/or frictional connection with the bone plate (2), characterized in that the bone plate (2), at least in a portion comprising the receiving opening (8), and the bone anchor (3), at least in a portion comprising the anchor head (6), are each made of a magnesium alloy, the magnesium alloy of the bone anchor (3) having a different hardness to the magnesium alloy of the bone plate (2).

2. The system according to claim 1, characterized in that the magnesium alloy of the bone anchor (3) has a higher hardness than the magnesium alloy of the bone plate (2).

3. The system according to claim 1, characterized in that the magnesium alloy with higher hardness has a material structure achieved at least in one step by cold forming.

4. The system according to claim 3, characterized in that the magnesium alloy with the higher hardness has a material structure achieved by hammering.

5. The system according to claim 1, characterized in that the magnesium alloys of the bone plate (2) on the one hand and of the bone anchor (3) on the other hand have the same chemical composition.

6. The system according to claim 1, characterized in that the magnesium alloy of the bone plate (2) and/or the magnesium alloy of the bone anchor (3) is/are resorbable.

7. The system according to claim 1, characterized in that the receiving opening (8) in an initial state does not have a connecting structure complementary to a connecting structure of the anchor head (6), or the anchor head (6) in an initial state does not have a connecting structure complementary to a connecting structure of the receiving opening (8).

8. The system according to claim 1, characterized in that the connecting structure of the anchor head (6) and/or the connecting structure of the receiving opening comprises or is a thread (7).

9. The system according to claim 7, characterized in that the receiving opening (8) in the initial state is thread-free or is provided with a thread (9) that is not complementary to the thread (7) of the anchor head (6), or the anchor head (6) in the initial state is thread-free or is provided with a thread (7) that is not complementary to the thread (9) of the receiving opening (8).

10. The system according to claim 1, characterized in that the bone anchor (3) has an anchor shaft (4) with a shaft thread (5).

11. The system according to claim 10, characterized in that a/the thread (7) of the anchor head (6) and the shaft thread (5) have different thread parameters.

12. The system according to claim 1, characterized in that there is a coating (12) on a main body made of the respective magnesium alloy on the bone plate (2), at least in a portion comprising the receiving opening (8), and/or on the bone anchor (3), at least in a portion comprising the anchor head (6).

13. The system according to claim 12, characterized in that the coating (12) comprises or is an oxide layer and/or a phosphate layer.

14. The system according to claim 1, characterized in that the receiving opening (8) and/or the anchor head (6) has/have a conical basic shape.

15. The system according to claim 14, characterized in that the receiving opening (8) and the anchor head (6) have conical basic shapes with different conicities.
